(19) United States
(12) Patent Application Publication
(10) Pub. No.: US 2020/0024313 A1
(43) Pub. Date: Jan. 23, 2020

(54) RECOMBINANT POLYPEPTIDE-ENRICHED CHLOROPLASTS OR ACCUMULATED LIPID PARTICLES AND METHODS FOR PRODUCING THE SAME IN ALGAE

(71) Applicant: Alcantara Research Group Inc., Calgary (CA)

(72) Inventor: Joenel Alcantara, Calgary (CA)

(21) Appl. No.: 16/513,514

(22) Filed: Jul. 16, 2019

Related U.S. Application Data
(60) Provisional application No. 62/699,894, filed on Jul. 18, 2018.

(51) Int. Cl. C07K 14/405; C12N 1/12; C12N 15/62; C12N 15/63

(52) U.S. Cl. CPC C07K 14/405; C12N 15/63; C12N 15/62; C12N 1/12

(57) ABSTRACT

The present disclosure relates to recombinant protein production in algal cells. In particular, the present disclosure provides methods for making recombinant polypeptides in association with accumulated lipid particles or chloroplasts. The methods involve producing the recombinant polypeptide as a fusion polypeptide with an oil body protein and the growth of the algal cells under non-homeostatic conditions to form accumulated lipid particles within the algal cells, wherein the algal lipid particles contain the fusion polypeptide.

### CROSS-REFERENCE TO RELATED APPLICATION

This application claims the benefit of and priority to U.S. Patent Application No. 62/699,894, filed on Jul. 18, 2018, and entitled “RECOMBINANT POLYPEPTIDE-ENRICHED CHLOROPLASTS OR ACCUMULATED LIPID PARTICLES AND METHODS FOR PRODUCING THE SAME IN ALGAE,” which is hereby incorporated by reference as if fully set forth herein.

### FIELD OF THE DISCLOSURE

The present disclosure pertains to the field of recombinant polypeptide production in algae, and in particular to methods for producing recombinant polypeptide-enriched chloroplasts and accumulated lipid particles.

### REFERENCE TO A SEQUENCE LISTING SUBMITTED AS A TEXT FILE VIA EFS WEB

This application includes a Sequence Listing in computer readable form, which is hereby incorporated by reference as if fully set forth herein. The Sequence Listing is provided in an ASCII text file named “2019-09-25 SeqListing_ST25”, which is 33 kb in size and was created on Sep. 25, 2019.

### BACKGROUND

A wide variety of techniques for the production of recombinant polypeptides in hosts are known in the art. Well-known examples include cell culture-based systems, such as microbial cell systems that use bacterial cells, fungal cells, or yeast cells, as well as animal cell systems including mammalian and insect cell culture systems. Other techniques for the production of polypeptides involve the generation of genetically modified plants and animals.

The benefits of using microbial cells to produce recombinant polypeptides include the low cost associated with cultivation of the cells, the potential for substantial product yields, and the general limited toxicity of raw materials. On a larger scale however, capital costs may become prohibitively expensive due to factors such as increased material requirements including growth media, scale-up of production facilities, and the expense associated with protein purification, notably in manufacturing operations designed to provide highly purified protein preparations, such as biopharmaceutical proteins.

Historically plants have represented an effective and economical method to produce recombinant polypeptides as they can be grown at a large scale with modest cost inputs. The use of plants has distinct advantages over bacterial systems as bacterial systems are frequently not appropriate for producing many eukaryotic proteins due to differences in protein processing and codon usage. Although foreign proteins have successfully been expressed in plants, the development of systems that offer commercially viable protein yields and are cost effective are still needed. One of the methods which has been explored is the method of production of recombinant polypeptides in association with plant oil-bodies as documented in for example U.S. Pat. No. 5,650,554.

Eukaryotic microalgae, hereinafter “algae” or “algal cells”, are eukaryotic photosynthetic organisms that can readily be grown in a variety of environments, such as large-scale bioreactors, making them attractive candidates for recombinant polypeptide expression.

Techniques to introduce genes capable of expressing recombinant polypeptides in algal cells are well known in the art and research efforts have been made to utilize algae for the purposes of the production of biomolecules as detailed in U.S. Pat. No. 8,951,777; U.S. Pat. No. 9,315,837; U.S. Patent Application No 2011/0030097; U.S. Patent Application No. 2012/0156717; U.S. Pat. No. 6,157,517 and PCT Patent Publication No. WO2012047970.

Algae in principle represent an attractive eukaryotic cellular host system for the synthesis of polypeptides due to the relative ease with which algal cells may be grown, as well as the availability of genetic engineering techniques. In many instances, upon production of the recombinant polypeptide, it is desirable to separate the polypeptide of interest from algal cellular constituents. Known techniques for the isolation of proteins from algal cells include the performance of a wide variety of protein purification techniques, such as chromatographical techniques, including ion exchange chromatography, high performance liquid chromatography, hydrophobic interaction chromatography, and the like. While these techniques are suitable to obtain substantially pure protein preparations on a laboratory scale, they are often inherently impractical to implement on the commercial scale. Moreover, commercial scale protein purification techniques are often the most expensive operational step. Due to the paucity of efficient protein production and extraction techniques known to the art, the commercial manufacture of proteins using algal cells remains substantially economically unviable.

Accordingly, there exists a need for improved techniques for the production of recombinant polypeptides in algae that are readily adaptable to commercial scale operations.

### SUMMARY

An object of the present disclosure is to provide recombinant polypeptide-enriched chloroplasts or Accumulated Lipid Particles (ALPs) and methods for producing the same in algae. In accordance with an aspect of the disclosure, there is provided a method of producing a recombinant polypeptide in an algal cell, the method comprising: (a) growing algal cells comprising a recombinant polypeptide under homeostatic conditions to target the recombinant polypeptide to the algal chloroplast; wherein the recombinant polypeptide is a fusion polypeptide comprising a steroleosin protein or fragment thereof; and (b) isolating the recombinant protein. In some embodiments, the method comprises isolating the algal chloroplasts and wherein the recombinant protein is isolated from the isolated algal chloroplasts. In some embodiments, the method comprises subjecting the growing algal cells to non-homeostatic conditions to form accumulated lipid particles within the algal cells, wherein the accumulated lipid particles comprise the fusion polypeptide and optionally isolating the accumulated lipid particles and optionally isolating the recombinant protein from the isolated accumulated lipid particles.

In accordance with one embodiment of the disclosure, there is provided recombinant polypeptide produced according to the method.

In accordance with one embodiment of the disclosure, there is provided a fusion polypeptide comprising a steroleosin protein or fragment and a polypeptide of interest.

In accordance with another aspect of the disclosure, there is provided a method of producing algal chloroplasts enriched for recombinant polypeptide, the method comprising: (a) introducing a nucleic acid into algal cells, the nucleic acid comprising as operably linked components (i) a nucleic acid encoding a fusion polypeptide comprising a steroleosin protein or fragment thereof to provide targeting to the algal chloroplast and a polypeptide of interest; and (ii) a nucleic acid sequence capable of controlling expression in an algal cell; (b) subjecting the algal cells in a growth medium to homeostatic conditions to target the fusion polypeptide to the algal chloroplast; and (c) optionally isolating the algal chloroplasts.

In accordance with one embodiment of the disclosure, there is provided algal chloroplasts isolated according to the method.

In accordance with another aspect of the disclosure, there is provided an algal cell comprising a fusion polypeptide comprising a steroleosin or fragment thereof and a protein of interest, wherein the steroleosin protein or fragment thereof targets the fusion polypeptide to chloroplasts when the algal cell is subjected to homeostatic conditions and to accumulated lipid particles when the algal cell is subjected to non-homeostatic conditions.

In accordance with another aspect of the disclosure, there is provided an algal cell comprising nucleic acid comprising as operably linked components (i) a nucleic acid sequence encoding fusion polypeptide comprising a steroleosin protein or fragment thereof and a protein of interest, wherein the steroleosin protein or fragment thereof targets the fusion polypeptide to chloroplasts when the cell is subjected to homeostatic conditions; and (ii) a nucleic acid sequence capable of controlling expression in an algal cell.

In accordance with another aspect of the disclosure, there is provided a preparation comprising chloroplasts wherein the chloroplasts comprise a fusion polypeptide comprising a steroleosin protein or fragment thereof and a protein of interest, wherein the steroleosin protein or fragment thereof targets the fusion polypeptide to chloroplasts when an algal cell is subjected to homeostatic conditions.

In accordance with another aspect of the disclosure, there is provided a preparation comprising accumulated lipid particles wherein the accumulated lipid particles comprise a fusion polypeptide comprising a steroleosin protein or fragment thereof and a protein of interest, wherein the steroleosin protein or fragment thereof targets the fusion polypeptide to accumulated lipid particles when the algal cell is subjected to non-homeostatic conditions.

In accordance with another aspect of the disclosure, there is provided a nucleic acid encoding a fusion polypeptide comprising a steroleosin protein or fragment thereof to provide targeting to algal chloroplast under homeostatic conditions and a polypeptide of interest.

In accordance with another aspect of the disclosure, there is provided a recombinant expression vector comprising a nucleic acid sequence encoding a fusion polypeptide comprising a steroleosin protein or fragment thereof to provide targeting to algal chloroplast under homeostatic conditions and to accumulated lipid particle under non-homeostatic conditions and a polypeptide of interest operatively linked to a nucleic acid sequence capable of controlling expression in an algal cell.

In accordance with another aspect of the disclosure, there is provided a method for producing accumulated lipid particles, the method comprising: (a) introducing a nucleic acid into algal cells, the nucleic acid comprising as operably linked components (i) a nucleic acid encoding a fusion polypeptide comprising a steroleosin protein or fragment thereof to provide targeting to the algal chloroplast under homeostatic conditions and accumulated lipid particles under non-homeostatic conditions and a polypeptide of interest; and (ii) a nucleic acid sequence capable of controlling expression in an algal cell; (b) subjecting the algal cell to non-homeostatic conditions to form accumulated lipid particles within the algal cell and (c) optionally isolating the accumulated lipid particles.

In accordance with one embodiment of the disclosure, there is provided accumulated lipid particles isolated according to the method.

In accordance with another aspect of the disclosure, there is provided a method of producing a recombinant polypeptide in an algal cell, the method comprising: culturing algal cell comprising fusion polypeptide comprising a steroleosin or fragment thereof and a protein of interest, wherein the steroleosin protein or fragment thereof targets the fusion polypeptide to chloroplasts when the cell is subjected to homeostatic conditions and accumulated lipid particles when the cell is subjected to non-homeostatic conditions and isolating the recombinant polypeptide.

Other features and advantages of the present disclosure will become apparent from the detailed description. It should be understood, however, that the detailed description, while indicating preferred implementations of the disclosure, are given by way of illustration only, since various changes and modifications within the spirit and scope of the disclosure will become apparent to those of skill in the art of the detailed description.

### BRIEF DESCRIPTION OF THE DRAWINGS

Embodiments of the disclosure will now be described, by way of example only, by reference to the attached Figure, wherein:

FIG. 1 show algal cells transformed with a plasmid that encodes a YFP recombinantly fused to a steroleosin protein under homeostatic (Ster+N) and non-homeostatic (Ster-N) conditions. Under homeostatic conditions (Ster+N), the fusion polypeptide is targeted to the chloroplast. Under non-homeostatic (removal of nitrogen from the growth media) conditions (Ster-N), fusion polypeptide is targeted to the ALPs.

### DETAILED DESCRIPTION

Definitions

Unless defined otherwise, all technical and scientific terms used herein have the same meaning as commonly understood by one of ordinary skill in the art.

The terms “accumulated lipid particle” or “ALP”, as may be used interchangeably herein, refer to subcellular sized approximately spherical particles comprising a triacycl glyceride core encapsulated by oil body proteins or chimeric proteins comprising fragments thereof produced under certain conditions in the transgenic algal cells of the disclosure.

The term “oil body protein” as used herein are proteins that are naturally associated with plant oil bodies and/or are naturally present on the phospholipid monolayer of plant oil bodies and includes steroleosins or functional fragments or derivatives thereof.

The terms “steroleosin”, “steroleosin protein” and “steroleosin polypeptide”, as used interchangeably herein, refer to any and all steroleosin polypeptides. An ordinarily skilled artisan would recognize that steroleosin polypeptides may be identified from publicly available databases. Polypeptides also include polypeptides comprising a sequence of amino acid residues which is (i) substantially identical to the amino acid sequences constituting any steroleosin polypeptides set forth herein in Table 1, or (ii) encoded by a nucleic acid sequence capable of hybridizing under at least moderately stringent conditions to the complement of a nucleic acid sequence encoding a steroleosin polypeptide set forth herein or a nucleic acid sequence encoding a steroleosin polypeptide set forth herein but for the use of synonymous codons.

The herein interchangeably used terms “nucleic acid sequence encoding a steroleosin”, “nucleic acid sequence encoding a steroleosin protein” and “nucleic acid sequence encoding a steroleosin polypeptide”, refer to any and all nucleic acid sequences encoding a steroleosin. An ordinarily skilled artisan would recognize that steroleosin polynucleotides may be identified from publicly available databases. Nucleic acids also include those having a sequence set forth in Table 1. Nucleic acid sequences encoding a steroleosin further include any and all nucleic acid sequences which (i) encode polypeptides that are substantially identical to the steroleosin amino acid sequences set forth herein; or (ii) hybridize to the complement of any steroleosin nucleic acid sequence set forth herein under at least moderately stringent hybridization conditions or which would hybridize thereto under at least moderately stringent conditions but for the use of synonymous codons.

By the term “substantially identical” it is meant that two polypeptide or polynucleotide sequences are at least 60% identical, and preferably are at least 85% identical and most preferably at least 95% identical, for example 96%, 97%, 98% or 99% identical.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.-16.6 (Log10 [Na+])+0.41(% (G+C)−600/l), or similar equation). Accordingly, the parameters in the wash conditions that determine hybrid stability are sodium ion concentration and temperature. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm (based on the above equation) -5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures. Additional guidance regarding hybridization conditions may be found in: Green and Sambrook, Molecular Cloning, a Laboratory Manual, Cold Spring Harbor Laboratory Press, 2012.

The term “homeostatic growth conditions” as used herein, in relation to the cultivation of algal cells, refers to growth conditions under which an algal cell culture is grown under substantially optimal growth conditions. Under homeostatic growth conditions algal cells in a cell culture can temporally exist in different growth phases, including a lag phase; a logarithmic growth phase, also known as exponential growth phase; a stationary phase; or a death phase. During each growth phase, algal cells have a characteristic growth rate corresponding with such growth phase when grown under homeostatic growth conditions. Notably, under homeostatic growth conditions, during logarithmic growth phase, the algal cell population doubles at a constant rate. The rate with which a cell population doubles in size is also known and referred herein as the doubling rate.

The terms “non-homeostatic growth conditions” and “non-homeostatic conditions”, as used herein in relation to the cultivation of algal cells, refers to conditions and growth conditions substantially deviating from homeostatic growth conditions. Under non-homeostatic growth conditions the algal growth rate substantially deviates from the corresponding growth rate under homeostatic growth conditions. When the conditions are altered during the logarithmic phase from homeostatic growth conditions to non-homeostatic conditions the doubling rate decreases to a doubling rate that is lower than the doubling rate during the logarithmic phase under homeostatic growth conditions.

### Overview

The present disclosure provides processes and methods of producing recombinant polypeptides in algal cells that are in association with lipid constituents of the cells, notably accumulated lipid particles or chloroplasts depending on growth conditions. By associating the recombinant polypeptides with the lipid constituents or chloroplasts, purification of the recombinant polypeptides may be facilitated since the accumulated lipid particles or chloroplasts and associated recombinant polypeptides can be readily separated from other cellular constituents prior to isolating the recombinant polypeptide. The methods of the disclosure accordingly eliminate the need for one or more conventional purification steps that are generally required when isolating recombinant polypeptides from algal cells.

The foregoing feature of the methods of the disclosure can facilitate scale-up and may allow for the economic production of recombinant polypeptides. In particular, the methods are amendable to the production of recombinant polypeptides both at laboratory scale and commercial scale.

As the recombinant polypeptide prior to targeting to the lipid constituents is synthesized in association with the chloroplasts, the recombinant protein is protected from degradation by cytoplasmic enzymes which may afford an advantage with respect to final polypeptide yield.

The method includes subjecting growing algal cells that express a steroleosin-fusion polypeptide to specific growth conditions depending on if the recombinant protein is to be targeted to accumulated lipid particles or chloroplasts.

In one embodiment, under homeostatic conditions the recombinant polypeptide is targeted to chloroplasts and under non-homeostatic conditions the recombinant polypeptide is targeted to accumulated lipid particles. The fusion polypeptide includes a steroleosin protein or fragment thereof that targets the fusion polypeptide to the lipid constituents and particularly accumulated lipid particles following stress or non-homeostatic conditions.

In some embodiments, more than one steroleosin protein or fragment thereof is used to target the protein of interest. In some embodiments, the algal cells may include more than one fusion protein each optionally have a different targeting steroleosin protein or fragment.

In some embodiments, the steroleosin protein or targeting fragment comprises fragments of more than one type of steroleosin protein and/or has been modified.

The recombinant polypeptide enriched accumulated lipid particles or chloroplasts may be isolated from the algae by various techniques known in the art. Optionally, the recombinant polypeptides are isolated from the accumulated lipid particles or chloroplasts. Techniques for isolating the polypeptides from the accumulated lipid particles or chloroplasts are also known in the art.

Alternatively, the recombinant polypeptide enriched accumulated lipid particles or chloroplasts are isolated for use in nutraceutical, pharmaceutical or other applications known in the art.

In addition to transforming algae with a single fusion construct, another embodiment of this disclosure is to transform different fusion constructs into the same algal cell either sequentially or simultaneously. For sequential transformation, the first fusion construct contains a selectable marker such as hygromycin resistance while the second fusion construct contains a different selectable marker such as norflurazon. The first construct can be transformed into algal cells such as Chlamydomonas reinhardtii by electroporation as previously described and then selected for by growing the electroporated algal cells on TAP agar plates containing hygromycin. After the algal cells have been identified to contain the first construct through resistance to the selectable marker and confirmed by PCR, the second fusion construct can be transformed by electroporation into algal cells containing the first construct and selecting for algal cells that can grow under TAP agar plates containing norflurazon. Presence of the second construct may also be confirmed via PCR. The algal cells transformed with both constructs are resistant to hygromycin and norflurazon and contain both the first and second fusion constructs. This same basic procedure can be applied to additional fusion constructs containing different selectable markers, such as zeocin, from previous constructs.

In some embodiments, a single vector encodes multiple fusion constructs.

In some embodiments, the recombinant protein enriched accumulated lipid particles or chloroplast may be orally ingested. Such encapsulation will protect the recombinant polypeptide from, for example, digestive processes that may degrade the polypeptide, preventing it from performing its biological function.

A worker skilled in the art would readily appreciate that the methods of the disclosure can be used with any or all eukaryotic, microalgal cells or algae including, without limitation any algae classified as green algae (Chlorophyceae), diatoms (Bacillariophyceae), yellow-green algae (Xanthophyceae), golden algae (Chrysophyceae), red algae (Rhodophyceae), brown algae (Phaeophyceae), dinoflagellates (Dinophyceae) or pico-plankton (Prasinophyceae and Eustigmatophyceae). Examples of algal cells further include any algal species belonging to the genus, Chlamydomonas, for example Chlamydomonas reinhardtii, and any algal species belonging to the genus Chiorella.

In one embodiment, the algal cell is a green algae (Chlorophyceae).

In one embodiment, the algal cell is a diatom (Bacillariophyceae).

In one embodiment, the algal cell is a yellow-green algae (Xanthophyceae).

In one embodiment, the algal cell is a golden algae (Chrysophyceae).

In one embodiment, the algal cell is a red algae (Rhodophyceae).

In one embodiment, the algal cell is a brown algae (Phaeophyceae).

In one embodiment, the algal cell is a dinoflagellates (Dinophyceae).

In one embodiment, the algal cell is a pico-plankton (Prasinophyceae and Eustigmatophyceae).

In one embodiment, the algal cell is an algal species belonging to the genus Clamydomonas, including but not limited to Chlamydomonas reinhardtii.

In one embodiment, the algal cell is an algal species belonging to the genus Chiorella.

In some embodiments, mixtures of eukaryotic algal species can be used, including but not limited to species belonging to any of the aforementioned.

In some embodiments, the algal cells are transgenic algae cells that are further modified. In some embodiments, the transgenic algae cells include a transgene, vector or like that is controlled by the recombinant protein of the disclosure.

### Recombinant Polypeptides and Polynucleotides

The present disclosure provides for recombinant polypeptides that can be targeted to accumulated lipid particles or chloroplasts in response to growth conditions.

Targeting to accumulated lipid particles or chloroplasts in response to growth conditions is a result of fusion of interest to a steroleosin protein. In certain embodiments, the targeting polypeptide is steroleosin, a derivative or fragment thereof. Non-limiting examples of steroleosin polypetide sequences that may be used include those set forth in Table 1.

In some embodiments, more than one steroleosin protein or fragment thereof is used to target the protein of interest. In some embodiments, the algal cells may include more than one fusion protein each optionally have a different targeting steroleosin protein or fragment.

In some embodiments, the steroleosin protein or targeting fragment comprises fragments of more than one type of steroleosin protein and/or has been modified.

In some embodiments, the targeting polypeptide includes substantially the full length steroleosin. In other embodiments, the targeting polypeptide excludes part of or all of the hydrophobic domain.

The present disclosure provides nucleic acid sequence encoding a fusion polypeptide comprising a portion of an oil body protein capable targeting of the fusion polypeptide to the algal chloroplast linked to a polypeptide of interest to lipid particles or chloroplasts in response to growth conditions. The nucleic acid may further include nucleic acid sequences capable of controlling expression in an algal cell.

In one embodiment, the nucleic acid encodes a sufficient portion of an oil body protein to provide targeting of the fusion polypeptide is an intact oil body protein, including an intact steroleosin. Non-limiting examples of nucleic acid sequences encoding steroleosins that may be used include the nucleic acids set forth in Table 1. Further oil body proteins that may be used in accordance herewith are steroleosins obtainable or obtained from an oil seed plant including, without limitation, thale cress (Arabidopsis thalania), soybean (Glycine max), rapeseed (Brassica spp.), sunflower (Heliantus annuus), safflower (Carthamus tinctorius, mustard (Brassica spp and Sinapis alba) and maize (Zea mays). In some embodiments, the nucleic acid sequences have been codon optimized for the specific algae.

In other embodiments, the nucleic acid encoding a sufficient portion of an oil body protein to provide targeting of the fusion polypeptide is a portion of an oil body protein, including a portion of a steroleosin.

In some embodiments, the portion of an oil body protein providing targeting of the fusion polypeptide comprises or consists of a proline knot motif.

In some embodiments, the portion of the oil body protein providing targeting comprises or consists of the central domain or a proline knot motif of a steroleosin and the N-terminal domain of a steroleosin.

In some embodiments, the portion of the oil body protein providing targeting comprises the N-terminal domain of a steroleosin and C-terminal domain of steroleosin.

The nucleic acid encoding a recombinant polypeptide may be any nucleic acid encoding a recombinant polypeptide, including any intact polypeptide of any length, varying from several amino acids in length to hundreds of amino acids in length, or any fragment or variant form of an intact recombinant polypeptide. In addition, in some embodiments, the nucleic acid encoding the polypeptide of interest may encode multiple polypeptides of interest, for example, a first and a second recombinant polypeptide, which may be linked to one another.

The recombinant polypeptide of interest may be any recombinant polypeptide including, without limitation insulin, hirudin, an interferon, a cytokine, an immunoglobulin, an antigenic polypeptide, a hemostatic factor, such as Willebrand Factor, a peptide hormone, such as angiotensin, β-glucuronidase (GUS), factor H binding protein, gam56, VP2, cellulase, xylanase, a protease, chymosin and chitinase.

As will readily be appreciated by those of skill in the art, depending on the nucleic acid sequence encoding the recombinant polypeptide, a wide variety of polypeptides may be selected and obtained, and the utility of the selected recombinant polypeptide may vary widely. Nucleic acid sequences encoding recombinant polypeptides may be identified and retrieved from databases such as GenBank (http://www.ncbi.nlm.nih.gov/genbank/) or nucleic acid sequences may be determined by methods such as gene cloning, probing and DNA sequencing. In accordance herewith, the nucleic acid sequence encoding the recombinant polypeptide may be selected in accordance with any and all applications for which the selected polypeptide is deemed useful. The actual nucleic acid sequence of the polypeptide of interest in accordance with the present disclosure is not limited, and may be selected as desired. In accordance herewith such recombinant polypeptides may be any polypeptides for use in pharmaceutical and biopharmaceutical or veterinary applications, any polypeptides for use in food, feed, nutritional and nutraceutical applications, any polypeptides for use in cosmetic and personal care applications, any polypeptides for use in agricultural applications, any polypeptides for use in industrial or domestic applications, any polypeptides that may be beneficial for algal growth, for example enzymes providing herbicidal or antibiotic resistance, and recombinant polypeptides for any other uses one desires to produce in accordance in accordance with the present disclosure.

In some embodiments, the 3′ end of the nucleic acid sequence encoding the sufficient portion of a polypeptide to provide targeting to an oil body is linked to the 5′ end of the nucleic acid sequence encoding the polypeptide of interest.

In some embodiments, the 5′ end nucleic acid sequence encoding the sufficient portion of a polypeptide to provide targeting to an oil body is linked to the 3′ end of the nucleic acid sequence encoding the polypeptide of interest.

In some embodiments, both the 5′ end and the 3′ end of the nucleic acid sequence encoding a sufficient portion of a polypeptide to provide targeting to an oil body are linked to the 3′ end of a nucleic acid sequence encoding the polypeptide of interest and to the 5′ end of a nucleic acid sequence encoding a polypeptide of interest, respectively. In this embodiment, the two recombinant polypeptides of interest may be identical or different.

In some embodiments, the 3′ end of a first nucleic acid sequence encoding a sufficient portion of a polypeptide to provide targeting of a fusion polypeptide is linked to the 5′ end of a nucleic acid sequence encoding a polypeptide of interest and the 3′ end of the same nucleic acid sequence encoding a polypeptide of interest is linked to the 5′ end of a second nucleic acid sequence encoding a sufficient portion of a polypeptide to provide targeting of to a fusion polypeptide.

In some embodiments, the nucleic acid sequence encoding a sufficient portion of an oil body protein to provide targeting to an oil body is separated from the nucleic acid sequence encoding by a cleavable peptide linker sequence. In some embodiments, the cleavable peptide linker sequence is enzymatically cleavable, for example a linker sequence cleavable by enzymes such as thrombin, Factor Xa collagenase, or chymosin. In other embodiments, the cleavable peptide linker sequence is chemically cleavable, for example cyanogen bromide. In further embodiments, the nucleic acid sequence further comprises a nucleic acid sequence that permits autocatalytic cleavage, for example, a nucleic acid sequence encoding a chymosin or an intein. Non-limiting examples of linkers are set forth in Table 1.

Nucleic acid sequences encoding fusion polypeptides can be prepared using any technique useful for the preparations of such nucleic acid sequences and generally involves obtaining a nucleic acid sequence encoding a sufficient portion of an oil body protein to target the fusion polypeptide, and a nucleic acid sequence encoding recombinant polypeptide of interest, for example by synthesizing these nucleic acid sequences, or isolating them from a natural source, and then linking the two nucleic acid sequences, using for example nucleic acid cloning vectors, such as the pUC and pET series of cloning vectors, microbial cloning host cells, such as Escherichia coli, and techniques such as restriction enzyme digestion, ligation, gel-electrophoresis, polymerase chain reactions (PCR), nucleic acid sequencing, and the like, which are generally known to those of skill in the art. Additional guidance regarding the preparation of nucleic acid sequences encoding fusion polypeptides including the use and cultivation of E. colias a microbial cloning host may be found in: Green and Sambrook, Molecular Cloning, a Laboratory Manual, Cold Spring Harbor Laboratory Press, 2012 and Esposito et al., 2009 Methods Mol. Biol. 498: 31-54.

In accordance with one aspect hereof, the nucleic acid sequence encoding a fusion polypeptide is linked to a nucleic acid sequence capable of controlling expression in an algal cell. Accordingly, the present disclosure also provides, in one embodiment, a nucleic acid sequence encoding a fusion polypeptide comprising a sufficient portion of an oil body protein to provide growth condition dependent targeting of the fusion polypeptide to accumulated lipid particles or chloroplast linked to a recombinant polypeptide; and a nucleic acid sequence capable of controlling expression in an algal cell.

In certain embodiments, the nucleic acid is optimized for expression in algae.

Nucleic acid sequences capable of controlling expression in algal cells that may be used herein include any transcriptional promoter capable of controlling expression of polypeptides in algal cells. Generally, promoters obtained from algal cells are used, including promoters associated with lipid production in algal cells. Promoters may be constitutive or inducible promoters, for example an oxygen inducible promoter. A non-limiting example of a transcriptional promoter that may be used in accordance herewith is set forth in Table 1. Further nucleic acid sequence elements capable of controlling expression in an algal cell include transcriptional terminators, enhancers and the like, all of which may be included in the chimeric nucleic acid sequences of the present disclosure.

In accordance with one aspect of the present disclosure, the nucleic acid comprising a nucleic acid sequence capable of controlling expression in algal cell linked to a nucleic acid sequence encoding a fusion polypeptide comprising a sufficient portion of steroleosin to provide targeting of the fusion polypeptide to an accumulated lipid particle linked to a recombinant polypeptide, can be integrated into a recombinant expression vector which ensures good expression in the algal cell.

The term “suitable for expression in an algal cell”, as used herein, means that the recombinant expression vector comprises the nucleic acid sequence of the present disclosure linked to genetic elements required to achieve expression in an algal cell. Genetic elements that may be included in the expression vector in this regard include a transcriptional termination region, one or more nucleic acid sequences encoding marker genes, one or more origins of replication and the like. The genetic elements are operably linked, typically as will be known to those of skill in the art, by linking e.g. a promoter in the 5′ to 3′ direction of transcription to a coding sequence. In preferred embodiments, the expression vector may further comprise genetic elements required for the integration of the vector or a portion thereof in the algal cell's genome.

Pursuant to the present disclosure, the expression vector can further contain a marker gene. Marker genes that may be used in accordance with the present disclosure include all genes that allow the distinction of transformed algal cells from non-transformed cells, including all selectable and screenable marker genes. A marker gene may be an antibiotic resistance marker against, for example, kanamycin, spectinomycin, hygromycin or zeocin. Further markers include herbicide resistance markers such as norflurazon, or metabolic markers that necessitate addition of substances to the media for growth such as in the case of arginine auxotrophy mutations. Screenable markers that may be employed to identify transformants through visual inspection include, β-galactosidase, β-glucuronidase (GUS) (U.S. Pat. Nos. 5,268,463 and 5,599,670), green fluorescent protein (GFP) (Niedz et al., 1995, Plant Cell Rep 14:403-406), and other fluorescent proteins.

To assemble the expression vector, an intermediary cloning host can be used. One intermediary cloning host cell that particularly conveniently can be used is E. coli using various techniques that are generally known to those of skill in the art including hereinbefore mentioned techniques for cloning and cultivation and general guidance that can for example be found in Sambrook et al., Molecular Cloning, a Laboratory Manual, Cold Spring Harbor Laboratory Press, 2001, Third Ed.

To introduce the chimeric nucleic acid sequence in algal cells, algal cells can be transformed using any technique known to the art, including, but not limited to, biolistic bombardment, glass beads, autolysin assisted transformation, electroporation, silicon carbide whiskers (Dunahay, T. G. (1993). BioTechniques 15, 452-460. Dunahay, T. G. , Adler , S. A., and Jarvik , J. W. (1997). Methods Mol. Biol. 62, 503-509), Agrobacterium-mediated gene transfer, and sonication or ultrasonication. The selected transformation technique can be varied depending on the algal species selected. In embodiments in which the selected algal cells lack a cell wall, glass bead transformation method is preferred. In the performance of this method, in general, glass beads containing the chimeric nucleic acid sequence, for example a linearized chimeric nucleic acid sequence, are placed in a reaction tube with an algae cell suspension and the mixture is vigorously vortexed to effect uptake of the chimeric nucleic acid sequence by the algal cells (Kindle, K. L., (1990). Proc. Natl. Acad. Sci (USA) 87, 1228 -1232). In embodiments in which the algal cells have cell walls, autolysin assisted transformation is a preferred methodology. In general, autolysin assisted transformation methodology, involves the incubation of algal cells with autolysin, an enzyme which naturally digests the cell wall during cellular mating and renders the algal cells susceptible to the receipt of nucleic acid material (Nelson et al., Mol. Cell Biol. 14: 4011-4019). In the performance of electroporation based techniques, an electric field is applied to the algal host cells to induce membrane permeability to effect uptake by the algal cells of the nucleic acid. Electroporation is a particularly preferred methodology since many algal species are readily susceptible to uptake of nucleic acid material upon electroporation (Brown et al., Mol. Cell Biol. (1991) 11 (4) 2382-2332 (PMC359944). In certain embodiments biolistic bombardment is used. In the performance of biolistic bombardment based techniques, in general, a particle delivery system is used to introduce the chimeric nucleic acid sequence into algae cells (Randolph-Anderson et al., BioRad Technical Bulletin no 2015 [http://www.bio-medicine. org/biology-technology/Sub-Micron-Gold-Particles-Are-Superior-to-Larger-Particles-for-Efficient-Biolistic-Transformation-of-Organelles-and-Some-Cells-1201-1/]. A further methodology that can be used to obtain transformed algal cells is Agrobacterium tumefaciens mediated transformation, which in general involves the infection of algal cells with Agrobacterium cells transformed to contain the chimeric nucleic acid sequence and upon infection transfer of the chimeric nucleic acid sequence to algal cells Kumar, S. V. et al. (2004). Genetic transformation of the green alga Chlamydomonas reinhardtii by Agrobacterium tumefaciens. Plant Sci. 166, 731-738. Yet one further methodology that in certain embodiments can be used is the use of ultrasound mediated delivery of the chimeric nucleic acid sequence into algae as is for example described in U.S. Patent Application no. US2015/0125960.

In some embodiments, upon introduction of the chimeric nucleic acid, the chimeric nucleic acid may be incorporated in the genome of the algal cell, generally resulted in inheritable expression. To facilitate integration in the genome of the algal cell, the chimeric nucleic acid may comprise one or more nucleic acid sequences that facilitate integration of the chimeric nucleic acid sequence in the algal genome.

In some embodiments, upon introduction of the chimeric nucleic acid, the chimeric nucleic acid may be maintained as a chimeric nucleic acid outside of the genome of the algal cell, generally resulting in transient expression.

### Growth Conditions

Under homeostatic growth conditions, the fusion protein is targeted to the chloroplasts. To target the fusion protein to the accumulated lipid particles from chloroplasts, the algal cells comprising the fusion proteins are subjected to stress or non-homeostatic growth condition.

In accordance with certain embodiments, the algal cells are grown in a growth medium under homeostatic growth conditions to target the recombinant polypeptide to the chloroplast within the algal cells.

In accordance with certain embodiments, the algal cells are grown in a growth medium under non-homeostatic growth conditions to form accumulated lipid particles within the algal cell, wherein the accumulated lipid particles comprise the fusion polypeptide.

In some embodiments, the algal cells are initially grown under homeostatic growth conditions wherein substantially no accumulated lipid particles are formed, and subsequently grown under non-homeostatic growth conditions.

Growth of algal cells under homeostatic conditions can be performed using any growth media suitable for the growth of algal cells, comprising non-limiting amounts of nutrients, including nutrients providing a carbon source, a nitrogen source, and a phosphorus source, as well as trace elements such as aluminum, cobalt, iron, magnesium, manganese, nickel, selenium zinc, and the like, and growing algal cells under optimal growth conditions. Conditions to achieve homeostatic growth for algal cells vary depending on the selected algal species, however such conditions typically include temperatures ranging, from 20° C. to 30° C., light intensities varying from 25-150 pE m⁻² s⁻¹ and a pH that is maintained in a range from 6.8 to 7.8.

Homeostatic growth conditions also include conditions appropriate for batch cultivation of algal cells, as well as conditions for continuous algal cell cultivation. In some embodiments, liquid culture media are used to grow the algal cells. In alternate embodiments, solid media for algal growth may also be used as a substrate for algal growth (The Chlamydomonas Sourcebook (Second Edition) Edited by: Elizabeth H. Harris, Ph. D., David B. Stern, Ph. D., and George B. Witman, Ph. D. ISBN: 978-0-12-370873-1) Further guidance to prepare suitable media for the homeostatic growth of algae, as well as guidance to suitable culturing conditions for algae are further described in Appl Microbiol Biotechnol. 2014 June; 98 (11):5069-79. doi: 10.1007/s00253-014-5593-y. Epub 2014 Mar. 4; Handbook of Microalgal Culture: Applied Phycology and Biotechnology By Amos Richmond, Qiang Hu ISBN 140517249; and in Algal Culturing Techniques Robert Arthur Anderson 2005 ISBN 0120884267. The concentration of a nutrient and/or a growth condition may be optimized or adjusted, for example by preparing a plurality of growth media, each including a different concentration of a nutrient, growing algal cells in each of the growth media, and evaluating algal growth, example, by evaluating cell density as a function of time. Then, a growth medium or growth condition can be selected that provides the most desirable effect.

In accordance with one aspect hereof, the algal cells are subjected to non-homeostatic conditions. Non-homeostatic conditions may be selected for the specific algae strain and/or selected based on the growth of the non-transgenic parent strain under those conditions. By “subjecting to non-homeostatic conditions”, it is meant that the conditions under which the algal cells are grown are gradually or abruptly modulated, or established in such a manner that algal cell growth rates substantially deviate from growth rates under homeostatic growth conditions. Thus, for example, the algal cell growth rate during log phase growth under homeostatic growth conditions deviates substantially from the algal cell growth rate during log phase growth under non-homeostatic conditions, and the algal cell growth rate during stationary phase growth under homeostatic growth conditions deviates substantially from the algal cell growth rate under non-homeostatic conditions. Substantial deviations include deviations wherein the growth rate under a non-homeostatic condition is less than about 0.8 or 0.8, about 0.7 or 0.7, about 0.6 or 0.6, about 0.5 or 0.5, about 0.4 or 0.4, about 0.3 or 0.3, about 0.2 or 0.2, or about 0.1 or 0.1 times the growth rate under a corresponding homeostatic growth condition.

In some embodiments, the algal cells immediately following introduction of the nucleic acid sequence within the algal cells are grown under non-homeostatic conditions. In some embodiments, the cells are grown or maintained in lag phase and not permitted to enter logarithmic phase.

In some embodiments, the algal cells, for example immediately following the introduction of the nucleic acid sequence, are initially grown under homeostatic conditions, and are then subjected to non-homeostatic conditions to grow or maintain the algal cells under non-homeostatic conditions.

In one embodiment, the algal cells are grown to logarithmic phase, and while in logarithmic phase the cells are subjected to non-homeostatic growth conditions to grow or maintain the algal cells under non-homeostatic conditions. Thus in this embodiment, the doubling rate decreases from a logarithmic doubling rate to a doubling rate that is substantially lower than the doubling rate under logarithmic homeostatic conditions, for example, the doubling rate under non-homeostatic conditions is less than about 0.8 or 0.8, about 0.7 or 0.7, about 0.6 or 0.6, about 0.5 or 0.5, about 0.4 or 0.4, about 0.3 or 0.3 , about 0.2 or 0.2, or about 0.1 or 0.1 times the doubling rate under homeostatic growth conditions during logarithmic phase. In some embodiments, the doubling rate, upon subjecting the cells to non-homeostatic conditions may alter from a constant doubling rate to a declining doubling rate.

In some embodiments, upon subjecting the cells to non-homeostatic conditions, the cells may enter a different growth phase, for example the cells may enter stationary growth phase from logarithmic phase.

In one embodiment, non-homeostatic growth conditions are conditions in which one or more nutrients are present in the algal cell growth medium in quantities that are insufficient for homeostatic algal cell growth.

In one embodiment, non-homeostatic growth conditions are conditions in which nitrogen is present in the algal cell growth medium in quantities that are insufficient for homeostatic algal cell growth. In some embodiments, the quantities of nitrogen present in the medium to for non-homeostatic growth ranges from about 0 mole/liter to about 0.02 mole/liter.

In one embodiment, non-homeostatic growth conditions are conditions in which phosphorus is present in the algal cell growth medium in quantities that are insufficient for homeostatic algal cell growth. In some embodiments, the quantities of phosphorus present in the medium for non-homeostatic growth ranges from about 0 to 0.8 mM.

In another embodiment, an exogenous stress factor, for example a physical, chemical or biological stress factor, is applied to an algal cell culture comprising a chimeric nucleic acid sequence of the present disclosure to effect non-homeostatic conditions.

In one embodiment, the exogenous stress factor applied is an adjustment of the pH of an algal cell culture to obtain a growth medium having non-homeostatic pH, and growing the cells at a non-homeostatic pH. In some embodiments, the pH is adjusted in such a manner that the pH of the algal culture ranges between about pH 5.0 to 6.5.

In one embodiment, the exogenous stress factor applied is an adjustment of the salinity of an algal cell culture to obtain a growth medium having a non-homeostatic salinity, and growing the cells under non-homeostatic salinity. In some embodiments, the salinity is adjusted in such a manner that the concentration of sodium and chloride ions of the algal culture ranges between about 20 to about 200 mM.

In one embodiment, the exogenous stress factor applied is an adjustment of the light intensity to which an algal cell culture is exposed to obtain a growth condition having a non-homeostatic light intensity, and growing the cells under non-homeostatic light intensity. In some embodiments, the light intensity is adjusted in such a manner that the light intensity to which the algal culture is exposed ranges between about 150-1000 pE m⁻² s⁻¹.

Non-homeostatic growth conditions may be detected and measured by comparing growth of algal cells under homeostatic conditions with growth of algal cells under non-homeostatic conditions. Thus, for example, the cell density of an algal cell culture may be determined, for example, by determining the optical density, or a cell counter such as a Coulter counter or flow cytometer, or manually counting cells using a hemocytometer, and the densities of algal cell cultures grown under homeostatic and non-homeostatic growth conditions may be compared. By measuring the cell density at different time points the growth rate and doubling rate of an algal cell culture, whether grown under homeostatic or non-homeostatic conditions, may be determined. Further guidance with respect to measuring algal cell growth may be found in The Chlamydomonas Sourcebook (Second Edition) Edited by: Elizabeth H. Harris, Ph. D., David B. Stern, Ph. D., and George B. Witman, Ph. D. (ISBN: 978-0-12-370873-1).

In accordance with one aspect, upon growth under non-homeostatic conditions, the algal cells produce accumulated lipid particles comprising a fusion polypeptide comprising the recombinant polypeptide.

In accordance with one embodiment, synthesis of the accumulated lipid particles produced by the algal cells when the cells are grown under non-homeostatic conditions, originate at the algal chloroplasts. Production of lipids, including in association with chloroplasts and accumulated lipid particles may be evaluated by staining algal cells with a lipophilic stain, such as Nile Red.

In accordance with one embodiment, the fusion polypeptide is upon introduction of the chimeric nucleic acid sequence in the algal cell first produced by the algal cell in association with the algal chloroplasts and thereafter, and upon subjecting the algal cells to non-homeostatic conditions, the fusion polypeptide is targeted to the accumulated lipid particles.

In accordance with one embodiment, hereof the fusion polypeptide is produced in association with the algal chloroplasts and the accumulated lipid particles and the fusion polypeptide is protected from exposure to the cytoplasm, and from degradation by cytoplasmic enzymes.

In some embodiments, targeting of the fusion polypeptide may be evaluated, for example using techniques such as electron microscopy, and confocal fluorescent microscopy in conjunction with fluorescent antibodies having a specificity for the recombinant polypeptide of interest.

In different embodiments, the algal cells may be subject to different non-homeostatic conditions, as herein before described, e.g. in the the presence of quantities of nutrients, such as nitrogen, or phosphate in quantities that are insufficient for homeostatic growth, or by subjecting the cells to an exogonous stress factor e.g. non-homeostatic pH conditions, non-homeostating light conditions or non-homeostatic salinity etc.

In some embodiments, the recombinant algal cells are grown at temperatures over 22° C. and with or without CO₂ over 0.5% to facilitate clumping.

In some embodiments, the clumping of recombinant algal cells may be facilitated by the addition of chemical additives to the media as is known in the art.

Harvesting

Recombinant protein can be isolated from either chloroplasts or from accumulated lipid particles depending on growth condition dependent targeting. In some embodiments, algal cells are grown under homeostatic conditions such that recombinant protein is targeted to chloroplasts. In some embodiments, algal cells are grown under non-homeostatic conditions prior harvesting such that recombinant protein is targeted to accumulated lipid particles.

Algal cells may be harvested by a variety of techniques known in the art including centrifugation and filtration. Optionally, harvesting includes a flocculation step where clumping of algal cells is promoted by growth conditions and/or additives and/or other methods known in the art.

In accordance with some embodiments where the harvesting of algal cells includes a flocculation step, the algal clumps are isolated.

In some embodiments after the algal cells are harvested, chloroplasts or accumulated lipid particles are isolated.

In accordance with one embodiment, chloroplasts comprising recombinant protein are isolated from the algal cells. Methodologies for the isolation of chloroplasts from algae will generally be known to those of skill in the art, and include but are not limited to the methodologies described in Mason, et al. (2006). Nat. Protoc. 1, 2227-2230.

In accordance with one embodiment, the accumulated lipid particles may be isolated. This may generally be accomplished by harvesting the algal cells by separating the algal cells from the growth medium. Thereafter the algal cells can be disrupted, using for example chemical techniques, such as enzymatic digestion, and/or physical techniques such as homogenization, sonication, and/or glass or ceramic beads to obtain a suspension comprising disrupted algal cells. The foregoing techniques are generally able to disrupt algal cell walls and membranes however they are relatively gentle in order to avoid compromising the integrity of the accumulated lipid particles. The accumulated lipid particles subsequently can readily be separated from other aqueous cell constituents.

In some embodiments, separation of the accumulated lipid particles is by density based separation techniques, for example centrifugation. Upon centrifugation of the suspension comprising disrupted algal cells a two-phase suspension comprising in a first phase the accumulated lipid particles and in a second phase the aqueous cellular constituents can be obtained from which the first phase comprising the accumulated lipid particles can be readily separated and removed. In some embodiments, accumulated lipid particles are isolated from cooled algal cells, optionally the algal cells cooled to 4° C.

The foregoing procedures may readily be conducted on a laboratory scale or on a larger commercial scale.

In some embodiments, the recombinant protein is isolated from the chloroplasts or accumulated lipid particles.

In accordance with one embodiment, the fusion polypeptide may include a cleavable linker sequence and upon isolation of the chloroplasts the recombinant polypeptide may be separated from the chloroplasts and the oil body protein, or portion thereof, as the case may be, and a substantially pure recombinant polypeptide may be obtained, using any protein purification methodology, including without limitation, those hereinbefore described.

The recombinant polypeptide may be recovered or isolated by a variety of different protein purification techniques including, e.g. metal-chelate chromatography, ion-exchange chromatography, size exclusion chromatography, affinity chromatography, hydrophobic interaction chromatography, reverse phase chromatography, gel filtration, etc. Further general guidance with respect to protein purification may for example be found in: Protein Purification: Principles, High Resolution Methods, and Applications, Janson, 2013, vol. 54. Wiley.

### EXAMPLES

Hereinafter are provided examples of specific implementations for performing the methods of the present disclosure, as well as implementations representing the compositions of the present disclosure. The examples are provided for illustrative purposes only, and are not intended to limit the scope of the present disclosure in any way.

### Vector Construction

The pChlamy_3 plasmid was obtained from Invitrogen. All standard recombinant DNA techniques (DNA digestion by restriction endonucleases, DNA ligation, plasmid isolation, and preparation of media and buffers) were performed as previously described (Sambrook, Fritsch, & Maniatis, 1989). The restriction endonucleases Kpnl and Xbal and T4 DNA ligase were from New England Biolabs.

### Transformation of Algal Cells

This method was used to transform a particular strain of Chlamydomonas reinhardtii via electroporation to introduce the steroleosin into the organism. The strains and culture conditions are as follows. Wild type C. reinhardtii cells of strain mt-[137c] were obtained from the Chlamydomonas Research Center (St. Paul, Minn.). Cells were grown at room temperature (RT) (22° C.) on a gyratory shaker at 120 rpm at a light intensity of 50 uE m-2 s-1 and a starting cell density of approximately 1.0×10⁵ cells/mL. Cells were grown in Tris-Acetate-Phosphate (TAP) culture medium (Harris, 1989).

Electroporation of Chlamydomonas cells with plasmid DNA was performed as previously described (Invitrogen, 2013). Briefly, 2 pg plasmid DNA was mixed in a 4 mm electroporation cuvette with 5.4×10⁴ wild type C. reinhardtii cells in exponential growth and incubated at room temperature for 5 minutes. After incubation, plasmid DNA was electroporated into Chlamydomonas cells with settings 50 uF, 1.5 kV cm-1, and infinite resistance. After electroporation, cells were resuspended in 12 mL of TAP+40 mM sucrose and incubated for 24 h at RT under white LED panels of intensity 50 uE m-2 s-1 with agitation of 100 rpm. After recovery, cells were centrifuged for 7 min at 1200 g, and resuspended in 750 μL TAP+40 mM sucrose. 250 ul of the cells were plated on each of three TAP+selection (10 μg/L hygromycin)+1.5% agar plates and incubated right side up at RT under white LED lights of 50 uE m-2 s-1 for 5 d or until colonies are clearly visible. Single colonies of at least 2 mm in diameter were used to inoculate TAP+10 μg/L hygromycin liquid media. Liquid cultures were incubated under standard growth conditions (50 uE m-2 s-1 from white LED panels, agitated on a gyratory shaker at 120 rpm, RT) until desired cell density was achieved, at which point cells could be subcultured.

### Production of ALPs in Algal Cells

ALPs were produced in algal cells by inoculating 50 mL of TAP media at a density of 1×10⁵ cells/mL, and growing to late log phase. Cultures to be nitrogen stressed (to be grown in TAP-N) were pelleted (1200 g, 5 min) and resuspended into an equal volume TAP-N medium (Siaut 2011, BMC Biotechnology 2011 Jan. 21; 11:7 doi 10.1186/1472-6750-11-7). Control cultures (to be grown in TAP+N) were pelleted (1200 g, 5 min) and resuspended in fresh TAP medium. All cultures were incubated 5 days after resuspension under standard growth conditions (50 μE m⁻² s⁻¹ from white LED panels, agitated at 100 rpm, at 22° C.) before imaging.

To image the ALPs produced in algal cells, 10 ul of the cultured cells of interest were transferred to a coated slide. The slide coat consisted of 2% agarose gel (Thermo Fisher Scientific) in TAP or TAP-N medium as appropriate and with the addition of 0.0001% w/v Nile Red (Sigma-Aldrich) when detection of triacylglycerides was desired. A 24×40 mm (No. 1 1/2) cover glass (Corning) was placed on top of the sample before the slide was subjected to analysis. An Olympus Fluoview FV10i (Olympus Canada Inc., Richmond Hill, Ontario) laser scanning confocal microscope was used to observe and capture images of the cells. All images were captured using an Olympus UPlanSApo 60× oil immersion objective (Olympus Canada Inc., Richmond Hill, Ontario). Additional digital magnification of 10× (total magnification of 600×) was applied using the Fluoview FV10i 1.2a software. Laser excitation and emission wavelengths for yellow fluorescent protein (YFP) were set to 480 nm and 527 nm respectively. Laser excitation and emission wavelengths for detection of triacylglycerides (TAGs) stained with Nile Red (NR) were set to 533 nm and 574 nm respectively. Where applicable, chloroplast autofluorescence (CHL) was imaged using an excitation wavelength of 473 nm and emission wavelength of 670 nm.

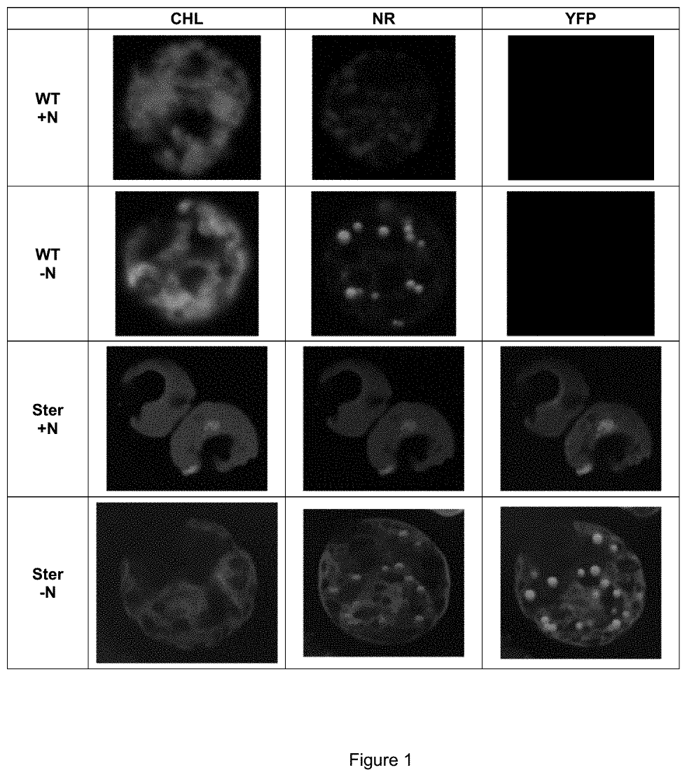

Referring to FIG. 1, Steroleosin fusion polypeptide is targeted to the chloroplast under homeostatic conditions while under stress (nitrogen depletion, TAP-N), the steroleosin fusion polypeptide is targeted to the ALPs.

SEQUENCE TABLE

| SEQ ID NO | SEQUENCE | NOTES |
|---|---|---|
| 1 | MDMLHTILN | Arabidopsis thaliana |
| | | N Terminal-Amino Acid Sequence |
| 2 | ATGGATATGCTTCACACTATTCTCAAT | Arabidopsis thaliana |
| | | N Terminal-Nucleic Acid |
| | | Sequence |
| 3 | ENVTGKVVLITGASSGIGEHVAYEYAKKGAKLALVARR | Arabidopsis thaliana- |
| | KDRLEIVAETSRQLGSGDVIIIPGDVSNVEDCKKFIDE | C-terminal-Amino Acid Sequence |
| | TIHHFGKLDHLINNAGVPQTVIFEDFTQIQDANSIMAS | |
| | KAALVKFFETLRVEISPDIKITIALPGFISTDMTTPQF | |
| | KEMYGSDFILSESVSRCAKAIFRGIGRGEAYVIEPSWI | |
| | KWIFLIKNVCPEIVDYLLDYIFVSYLKPYFKRD | |
| 4 | GAAAACGTTACTGGGAAAGTAGTTCTAATCACCGGAGC | Arabidopsis thaliana- |
| | TTCCTCCGGCATCGGAGAGCATGTGGCATACGAGTACG | C term-Nucleic Acid Seq |
| | CAAAGAAAGGAGCAAAATTAGCTCTTGTTGCTCGAAGA | |
| | AAGGATCGTCTAGAGATTGTAGCAGAAACATCACGTCA | |
| | ATTAGGATCTGGCGATGTCATCATCATACCTGGTGATG | |
| | TTTCTAATGTTGAGGACTGCAAGAAGTTCATCGACGAA | |
| | ACGATTCATCATTTCGGAAAACTGGACCACCTGATCAA | |
| | CAATGCTGGAGTACCTCAAACTGTTATATTCGAAGATT | |
| | TCACACAAATTCAAGACGCTAATTCTATAATGGCAAGC | |
| | AAAGCAGCTTTAGTAAAATTCTTTGAAACATTAAGAGT | |
| | TGAGATCAGTCCCGATATCAAAATAACAATCGCTCTTC | |
| | CCGGATTTATATCTACGGATATGACCACTCCTCAGTTT | |
| | AAAGAAATGTATGGTTCAGATTTCATACTTTCGGAATC | |
| | AGTGAGTCGATGCGCAAAAGCGATCTTCAGAGGTATTG | |
| | GTAGAGGAGAGGCATACGTCATAGAGCCATCTTGGATA | |
| | AAATGGATCTTTTTGATAAAAAATGTTTGTCCTGAGAT | |
| | CGTTGACTACTTACTCGACTATATATTTGTTAGTTATC | |
| | TAAAACCTTATTTCAAGCGTGAT | |
| 5 | MEQVVNAVLD | Oryza sativa N Terminal- |
| | | Amino Acid Sequence |
| 6 | ATGGAGCAGGTGGTGAACGCGGTGCTGGAC | Oryza sativa N Terminal- |
| | | Nucleic Acid Sequence |
| 7 | ENMHNKVVLITGASSAIGEQIAYEYARRNANLVLVARR | Oryza sativa C Terminal- |
| | EHRLFAVRENARALGAGQVLVIAADVVKEDDCRRLVGD | Amino Acid Sequence |
| | TISFFGQLNHLVNTVSLGHDFCFEEAGDTVAFPHLMDV | |
| | NFWGNVYPTYAALPYLRRSHGRVVVNAAVEIWLPMPRM | |
| | TLYSAAKAAVIDFYESLRYEVGDEVGISVATHGWIGGE | |
| | ASGGKFMLEEGAEMQWKGEEREVPLAGGQVEAYARMVV | |
| | AGACRGDAHVKHPNVVYDVFLVFRAFAPDVLAVVTFRL | |
| | LLSTPSPSPPASARRHQLAALPAPPLHPLLEYPSARSP | |
| | GRAAQQHKLE | |
| 8 | GAGAACATGCACAACAAGGTCGTCCTCATCACCGGCGC | Oryza sativa C Terminal- |
| | CTCCTCCGCCATCGGAGAGCAAATCGCGTACGAGTACG | Nucleic Acid Sequence |
| | CGCGGAGGAACGCGAACCTGGTGCTGGTGGCGAGGCGG | |
| | GAGCACCGGCTGTTCGCGGTGCGGGAGAACGCGCGGGC | |
| | CCTCGGCGCCGGCCAGGTCCTCGTCATCGCCGCCGACG | |
| | TCGTCAAGGAGGACGACTGCCGGAGGCTCGTCGGCGAC | |
| | ACCATCAGCTTCTTCGGCCAGTTAAACCATCTGGTGAA | |
| | CACGGTGAGCTTGGGCCACGATTTCTGCTTCGAGGAGG | |
| | CCGGCGACACGGTCGCGTTCCCTCACCTCATGGACGTC | |
| | AACTTCTGGGGGAACGTGTACCCGACGTACGCCGCGCT | |
| | CCCCTACCTGCGGCGGAGCCATGGCCGTGTCGTCGTCA | |
| | ACGCCGCCGTCGAGATCTGGCTGCCCATGCCCAGGATG | |
| | ACCCTCTACTCTGCGGCCAAGGCGGCGGTGATCGATTT | |
| | CTACGAGTCGCTCCGGTACGAGGTGGGCGATGAGGTGG | |
| | GCATCAGCGTGGCGACGCACGGCTGGATCGGCGGCGAG | |
| | GCCAGCGGCGGCAAGTTCATGCTCGAGGAAGGCGCAGA | |
| | GATGCAATGGAAAGGGGAGGAGCGAGAGGTGCCGCTCG | |
| | CCGGCGGGCAAGTGGAGGCGTACGCGAGGATGGTGGTC | |
| | GCCGGCGCGTGCCGCGGCGACGCCCACGTGAAGCACCC | |
| | CAACTGGTACGACGTCTTCCTTGTCTTCCGCGCCTTCG | |
| | CGCCCGACGTCCTCGCCTGGACGTTCCGCCTCCTGCTG | |
| | TCCACGCCATCGCCGTCGCCGCCGGCGAGCGCCCGTCG | |
| | CCACCAGCTCGCCGCGCTGCCGGCTCCGCCGCTCCACC | |
| | CGCTGCTCGAGTACCCGTCGGCTCGGAGCCCCGGGCGC | |
| | GCCGCCCAGCAGCACAAGCTGGAG | |
| 9 | MSFINSVLD | Populus trichocarpa N |
| | | Terminal-Amino Acid Sequence |
| 10 | ATGAGTTTTATAAACTCTGTGTTGGAT | Populus trichocarpa N Terminal- |
| | | Nucleic Acid Sequence |
| 11 | EDMEDKVVIITGASSGIGEQIAYEYAKRKAILVLIARR | Populus trichocarpa C Terminal- |
| | EHRLRGVSEKARYIGAKRVLIMAADVVKEDDCRRFVNE | Amino Acid Sequence |
| | TINYFGRVDHLVNTASLGHTFYFEEVGDTSVFPHFLDI | |
| | NFWGNVYPTYVALPYLRQSNGRVVVNAAVESWLPLPRM | |
| | SLYAAAKAALVSFYESLRFEVNGEVGITIASHGWIGSE | |
| | MSRGKFMLEDGAEMQWKEEREVNGTGGPVEDYAKMIVS | |
| | GACRGHQYVKYPSWYDIFLLYRMFAPGILNWALRMLLA | |
| | PNGSRRTSMIGTGRPALI | |
| 12 | GAGGATATGGAGGATAAAGTTGTTATCATCACTGGAGC | Populus trichocarpa C Terminal- |
| | TTCTTCTGGCATAGGAGAACAAATTGCATATGAATATG | Nucleic Acid Sequence |
| | CAAAGAGGAAAGCAATTCTTGTTCTGATTGCACGTAGA | |
| | GAGCACCGGCTTAGAGGGGTCAGTGAGAAAGCTAGGTA | |
| | TATTGGTGCAAAGCGTGTCCTGATTATGGCTGCAGATG | |
| | TTGTCAAGGAGGATGATTGTAGGAGATTTGTCAATGAG | |
| | ACCATAAATTACTTTGGTCGGGTGGATCATCTTGTCAA | |
| | TACAGCAAGTTTGGGGCATACATTTTACTTTGAAGAAG | |
| | TAGGAGACACCTCTGTGTTTCCCCATTTCTTGGACATA | |
| | AACTTTTGGGGAAATGTCTATCCAACTTATGTGGCTCT | |
| | TCCATACCTACGTCAGAGCAATGGACGAGTTGTTGTTA | |
| | ATGCAGCAGTTGAGAGCTGGTTACCTCTGCCGAGAATG | |
| | AGCTTATATGCTGCTGCAAAGGCTGCCCTGGTGAGCTT | |
| | CTACGAGTCACTGAGATTTGAAGTGAATGGTGAAGTTG | |
| | GAATAACAATTGCATCTCATGGTTGGATTGGGAGCGAA | |
| | ATGAGTAGAGGCAAGTTCATGCTAGAGGATGGAGCAGA | |
| | GATGCAATGGAAAGAAGAGAGAGAAGTAAACGGGACTG | |
| | GTGGTCCAGTAGAGGACTATGCAAAGATGATTGTGTCG | |
| | GGAGCTTGCCGAGGACATCAATATGTCAAGTACCCAAG | |
| | CTGGTATGACATATTCCTCCTTTACAGGATGTTTGCAC | |
| | CTGGAATTCTCAACTGGGCTCTTCGAATGTTGCTTGCA | |
| | CCGAATGGTTCAAGAAGAACGTCTATGATAGGCACCGG | |
| | GAGACCTGCATTAATT | |
| 13 | MDLIQMLLN | Selaginella moellendorffii |
| | | N Terminal-Amino Acid |
| | | Sequence |
| 14 | ATGGATTTGATACAAATGCTGCTTAAT | Selaginella moellendorffii |
| | | N Terminal-Nucleic Acid |
| | | Sequence |
| 15 | ENVRGKVAVITGASSGIGEYMAYEYGKRGAKVVLCGRR | Selaginella moellendorffii |
| | ENQLKNVQERVGSEGATDTLVVVADVSREEECKKVVDE | C Terminal-Amino Acid |
| | TINTLGKIDHLVCNHGIANSFFVEEAKGLEIFRKIMDV | Sequence |
| | NFMGCVYTTYFALPHLRKSRGKIVVTASTASWLPIPRM | |
| | SIYNASKAAVVNFFDTLRTELRSDIGGMTIAMPGYIHS | |
| | EMTMGKFMSAEGKHDMNVDIRDTLVGPSPVASTQYCAK | |
| | QIVSAVTRGERYVVVPTVVYKVSLLFRVFVPQLLETFI | |
| | SLLFVKELQPGKPVTKVIMDSFPSAEKVLYPPGMQQKT | |
| | D | |
| 16 | GAAAATGTCCGCGGCAAGGTCGCCGTCATCACTGGGGC | Selaginella moellendorffii |
| | TTCTTCCGGGATTGGAGAGTACATGGCTTATGAGTATG | C Terminal-Nucleic Acid |
| | GCAAGCGTGGAGCCAAGGTGGTGCTTTGTGGGAGGCGA | Sequence |
| | GAGAATCAACTCAAGAACGTCCAGGAGCGCGTTGGATC | |
| | CGAGGGTGCTACTGATACTCTGGTGGTTGTCGCCGACG | |
| | TTTCTAGGGAGGAAGAATGTAAGAAGGTTGTGGATGAG | |
| | ACGATCAACACCTTGGGAAAAATTGATCACCTTGTCTG | |
| | CAATCATGGTATTGCGAACAGCTTCTTCGTCGAGGAAG | |
| | CGAAAGGTTTGGAGATCTTCCGCAAGATCATGGATGTG | |
| | AATTTCATGGGTTGCGTCTATACTACATACTTCGCTTT | |
| | GCCTCATCTCCGCAAGAGCCGAGGAAAGATTGTCGTCA | |
| | CGGCCTCGACTGCGTCCTGGCTTCCCATCCCGAGGATG | |
| | TCAATCTACAACGCCTCCAAAGCAGCCGTGGTGAACTT | |
| | CTTCGATACCCTGAGGACTGAGCTTCGATCAGATATAG | |
| | GAGGAATGACGATTGCCATGCCTGGCTACATCCACAGC | |
| | GAGATGACAATGGGCAAGTTCATGTCAGCCGAGGGGAA | |
| | GCACGACATGAACGTTGACATACGCGACACTCTCGTCG | |
| | GCCCTTCTCCAGTAGCTTCAACGCAGTACTGCGCGAAG | |
| | CAGATCGTGTCCGCGGTCACCAGGGGCGAGCGCTACGT | |
| | GGTGGTGCCGACCTGGTACAAGGTCTCACTCCTGTTTC | |
| | GCGTCTTCGTTCCCCAGCTCCTGGAGACTTTCATAAGC | |
| | CTCTTGTTCGTCAAGGAGCTACAGCCAGGGAAGCCAGT | |
| | CACCAAAGTCATCATGGACAGCTTCCCGAGCGCCGAGA | |
| | AAGTCTTGTATCCCCCTGGAATGCAGCAGAAGACAGAC | |
| 17 | MGFLGSFLN | Physcomitrella patens |
| | | N Terminal-Amino Acid Sequence |
| 18 | ATGGGATTCCTTGGAAGCTTCCTCAAC | Physcomitrella patens |
| | | N Terminal-Nucleic Acid |
| | | Sequence |
| 19 | EDMRGKVVLITGASSGIGEHIAYEYAKKGARLALVGRR | Physcomitrella patens |
| | ENLLMEVADRAITRGASDVKVLVGDVTKEADCKRFLEE | C Terminal-Amino Acid Sequence |
| | TIQKYGRLDHLVNNAGVAHSFFFSETKDLKALTSTLDT | |
| | TFWGQVYMTYFAIPHLRRTHGKVLVMASTASWLPYPRQ | |
| | TLYNAGKAGVLAFFDTLRVEVGDVIGITIVMPGWIESE | |
| | ITKGKFIHEDGDIVVTDQMERDMHIGPVPVTSVTECAN | |
| | AAVKGVIRGSHYVTVPFYYSAFLLYRMFAPEVLDWIFR | |
| | LIFVKHPQKPLSKQVLKASEVHRVLYPTSIQKAD | |
| 20 | GAGGACATGCGAGGAAAAGTCGTACTAATCACAGGAGC | Physcomitrella patens |
| | ATCATCTGGAATTGGAGAGCACATAGCGTATGAGTACG | C Terminal-Nucleic Acid |
| | CGAAGAAGGGGGCTCGCCTCGCGCTCGTGGGACGGCGG | Sequence |
| | GAGAATTTGCTCATGGAGGTTGCAGACAGGGCGATAAC | |
| | GAGAGGGGCATCAGATGTGAAGGTCTTAGTCGGAGATG | |
| | TCACGAAAGAGGCAGACTGCAAACGTTTTCTAGAAGAA | |
| | ACGATTCAAAAATATGGCCGGCTGGACCATCTGGTGAA | |
| | CAACGCTGGAGTTGCTCACAGCTTCTTCTTTAGCGAGA | |
| | CAAAGGATTTGAAGGCGCTGACATCAACTTTGGACACA | |
| | ACATTCTGGGGTCAAGTGTACATGACGTACTTTGCAAT | |
| | TCCGCATTTGCGGCGCACACATGGCAAGGTTCTCGTTA | |
| | TGGCGTCTACTGCGAGCTGGCTGCCATATCCACGGCAA | |
| | ACACTATACAATGCTGGAAAGGCAGGTGTGTTAGCTTT | |
| | CTTTGACACATTACGAGTAGAGGTTGGTGACGTAATTG | |
| | GTATCACCATAGTCATGCCGGGATGGATTGAAAGCGAG | |
| | ATCACTAAAGGCAAATTTATACATGAAGATGGCGACAT | |
| | ATGGACAGACCAGATGGAACGAGACATGCATATCGGTC | |
| | CAGTGCCCGTGACATCGGTAACTGAATGTGCCAACGCT | |
| | GCAGTGAAGGGCGTTATTAGAGGCTCCCATTATGTGAC | |
| | GGTTCCATTTTACTACTCTGCGTTTCTCTTATACCGAA | |
| | TGTTTGCTCCTGAAGTCTTGGATTGGATATTCCGACTT | |
| | ATTTTTGTTAAACATCCCCAGAAGCCTCTGAGCAAACA | |
| | GGTACTGAAAGCCTCAGAGGTTCACAGGGTTTTGTACC | |
| | CAACAAGTATTCAGAAGGCAGAT | |
| 21 | MELINDFLNLTAPFFTFFGLCFFLPPFYFFKFLQSIFS | Arabidopsis thaliana |
| | TI | Hydrophobic Domain Amino |
| | | Acid-Accession NM_124448 |
| 22 | ATGGAGTTGATAAACGACTTTCTCAACCTAACTGCTCC | Arabidopsis thaliana |
| | TTTCTTCACCTTCTTTGGTCTCTGCTTCTTCTTGCCTC | Hydrophobic Domain Nucleic |
| | CTTTTTACTTCTTCAAGTTCTTGCAGTCTATTTTCTCG | Acid-Accession NM_124448 |
| | ACAATT | |
| 23 | MDLIHTFLNLIAPPFTFFFLLFFLPPFQIFKFFLSILG | Sesamum indicum |
| | TL | Hydrophobic Domain Amino |
| | | Acid-Accession AF302806 |
| 24 | ATGGATCTAATCCACACTTTCCTCAACTTAATAGCTCC | Sesamum indicum |
| | CCCTTTCACCTTCTTCTTCCTTCTCTTTTTCTTGCCAC | Hydrophobic Domain Nucleic |
| | CCTTCCAGATTTTCAAGTTCTTCCTTTCAATCTTGGGC | Acid-Accession AF302806 |
| | ACCCTT | |
| 25 | M E L I N D F L N L T A P F F T F F G | Arabidopsis thaliana |
| | L C F F L P P F Y F F K F L Q S I F S | steroleosin protein sequence |
| | T I F S E N L Y G K V V L I T G A S S | |
| | G I G E Q L A Y E Y A C R G A C L A L | |
| | T A R R K N R L E E V A E I A R E L G | |
| | S P N V V T V H A D V S K P D D C R R | |
| | I V D D T I T H F G R L D H L V N N A | |
| | G M T Q I S M F E N I E D I T R T K A | |
| | V L D T N F W G S V Y T T R A A L P Y | |
| | L R Q S N G K I V A M S S S A A W L T | |
| | A P R M S F Y N A S K A A L L S F F E | |
| | T M R I E L G G D V H I T I V T P G Y | |
| | I E S E L T Q G K Y F S G E G E L I V | |
| | N Q D M R D V Q V G P F P V A S A S G | |
| | C A K S I V N G V C R K Q R Y V T E P | |
| | S W F K V T Y L W K V L C P E L I E W | |
| | G C R L L Y M T G T G M S E D T A L N | |
| | K R I M D I P G V R D Y K D D D D K E | |
| | N L Y F | |
| 26 | ENLYFQS | Synthetic Linker |
| 27 | GAGAACCTCTACTTCCAATCG | Synthetic linker |
| 28 | CITGDALVALPEGESVRIADIVPGARPNSDNAIDLKVL | Synthetic Linker (intein) |
| | DRHGNPVLADRLFHSGEHPVYTVRTVEGLRVTGTANHP | |
| | LLCLVDVAGVPTLLWKLIDEIKPGDYAVIQRSAFSVDC | |
| | AGFARGKPEFAPTTYTVGVPGLVRFLEAHHRDPDAQAI | |
| | ADELTDGRFYYAKVASVTDAGVQPVYSLRVDTADHAFI | |
| | TNGFVSHA | |
| 29 | TGCATCACGGGAGATGCACTAGTTGCCCTACCCGAGGG | Synthetic linker (Intein) |
| | CGAGTCGGTACGCATCGCCGACATCGTGCCGGGTGCGC | |
| | GGCCCAACAGTGACAACGCCATCGACCTGAAAGTCCTT | |
| | GACCGGCATGGCAATCCCGTGCTCGCCGACCGGCTGTT | |
| | CCACTCCGGCGAGCATCCGGTGTACACGGTGCGTACGG | |
| | TCGAAGGTCTGCGTGTGACGGGCACCGCGAACCACCCG | |
| | TTGTTGTGTTTGGTCGACGTCGCCGGGGTGCCGACCCT | |
| | GCTGTGGAAGCTGATCGACGAAATCAAGCCGGGCGATT | |
| | ACGCGGTGATTCAACGCAGCGCATTCAGCGTCGACTGT | |
| | GCAGGTTTTGCCCGCGGGAAACCCGAATTTGCGCCCAC | |
| | AACCTACACAGTCGGCGTCCCTGGACTGGTGCGTTTCT | |
| | TGGAAGCACACCACCGAGACCCGGACGCCCAAGCTATC | |
| | GCCGACGAGCTGACCGACGGGCGGTTCTACTACGCGAA | |
| | AGTCGCCAGTGTCACCGACGCCGGCGTGCAGCCGGTGT | |
| | ATAGCCTTCGTGTCGACACGGCAGACCACGCGTTTATC | |
| | ACGAACGGGTTCGTCAGCCACGCT | |
| 30 | TCGCTGAGGCTTGACATGATTGGTGCGTATGTTTGTAT | Synthetic-promoter Hsp70A- |
| | GAAGCTACAGGACTGATTTGGCGGGCTATGAGGGCGGG | Rbc52 |
| | GGAAGCTCTGGAAGGGCCGCGATGGGGCGCGCGGCGTC | |
| | CAGAAGGCGCCATACGGCCCGCTGGCGGCACCCATCCG | |
| | GTATAAAAGCCCGCGACCCCGAACGGTGACCTCCACTT | |
| | TCAGCGACAAACGAGCACTTATACATACGCGACTATTC | |
| | TGCCGCTATACATAACCACTCAGCTAGCTTAAGATCCC | |
| | ATCAAGCTTGCATGCCGGGCGCGCCAGAAGGAGCGCAG | |
| | CCAAACCAGGATGATGTTTGATGGGGTATTTGAGCACT | |
| | TGCAACCCTTATCCGGAAGCCCCCTGGCCCACAAAGGC | |
| | TAGGCGCCAATGCAAGCAGTTCGCATGCAGCCCCTGGA | |
| | GCGGTGCCCTCCTGATAAACCGGCCAGGGGGCCTATGT | |
| | TCTTTACTTTTTTACAAGAGAAGTCACTCAACATCTTA | |
| | AA | |

### REFERENCES

Umate, Genomics Proteomics Bioinformatics 10(2012) 345-353.
Lin et al. (2002) Plant physiology 128(4):1200-11·May 2002

What is claimed is:

1. A method of producing a recombinant polypeptide in an algal cell, the method comprising:
growing algal cells comprising a recombinant polypeptide under homeostatic conditions to target the recombinant polypeptide to the algal chloroplast; wherein the recombinant polypeptide is a fusion polypeptide comprising a steroleosin protein or fragment thereof and a protein of interest; and isolating the recombinant polypeptide.

2. The method of claim 1, comprising isolating the algal chloroplasts and wherein the recombinant polypeptide is isolated from the isolated algal chloroplasts.

3. The method of claim 1, comprising subjecting the growing algal cells to non-homeostatic conditions to form accumulated lipid particles within the algal cells, wherein the accumulated lipid particles comprise the fusion polypeptide.

4. The method of claim 3, comprising isolating the accumulated lipid particles and wherein the recombinant protein is isolated from the isolated accumulated lipid particles.

5. The method of claim 1, comprising the step of introducing a nucleic acid encoding the recombinant polypeptide into the algal cell.

6. The method of claim 3, wherein non-homeostatic conditions include a deficiency in one or more nutrients.

7. The method of claim 6, wherein the nutrient is nitrogen.

8. A method of producing algal chloroplasts enriched for recombinant polypeptide, the method comprising:
(a) introducing a nucleic acid into algal cells, the nucleic acid comprising as operably linked components
(i) a nucleic acid encoding a fusion polypeptide comprising a steroleosin protein or fragment thereof to provide targeting to the algal chloroplast and a polypeptide of interest; and
(ii) a nucleic acid sequence capable of controlling expression in an algal cell;
(b) subjecting the algal cells in a growth medium to homeostatic conditions to target the fusion polypeptide to the algal chloroplast; and
(c) optionally isolating the algal chloroplasts.

9. A method for producing accumulated lipid particles, the method comprising:
(a) introducing a nucleic acid into algal cells, the nucleic acid comprising as operably linked components
a nucleic acid encoding a fusion polypeptide comprising a steroleosin protein or fragment thereof to provide targeting to the algal chloroplast and a polypeptide of interest; and
a nucleic acid sequence capable of controlling expression in an algal cell;
(b) subjecting the algal cell to non-homeostatic conditions to form accumulated lipid particles within the algal cell.


SEQUENCE LISTING (30 sequences)

SEQ ID NO: 1 - protein, 9 aa, Arabidopsis thaliana
MDMLHTILN

SEQ ID NO: 2 - DNA, 27 bp, Arabidopsis thaliana
atggatatgcttcacactattctcaat

SEQ ID NO: 3 - protein, 223 aa, Arabidopsis thaliana
ENVTGKVVLITGASSGIGEHVAYEYAKKGAKLALVARRKDRLEIVAETSRQLGSGDVIIIPGDVSNVEDCKKFIDETIHHFGKLDHLINNAGVPQTVIFEDFTQIQDANSIMASKAALVKFFETLRVEISPDIKITIALPGFISTDMTTPQFKEMYGSDFILSESVSRCAKAIFRGIGRGEAYVIEPSWIKWIFLIKNVCPEIVDYLLDYIFVSYLKPYFKRD

SEQ ID NO: 4 - DNA, 669 bp, Arabidopsis thaliana-C
gaaaacgttactgggaaagtagttctaatcaccggagcttcctccggcatcggagagcatgtggcatacgagtacgcaaagaaaggagcaaaattagctcttgttgctcgaagaaaggatcgtctagagattgtagcagaaacatcacgtcaattaggatctggcgatgtcatcatcatacctggtgatgtttctaatgttgaggactgcaagaagttcatcgacgaaacgattcatcatttcggaaaactggaccacctgatcaacaatgctggagtacctcaaactgttatattcgaagatttcacacaaattcaagacgctaattctataatggcaagcaaagcagctttagtaaaattctttgaaacattaagagttgagatcagtcccgatatcaaaataacaatcgctcttcccggatttatatctacggatatgaccactcctcagtttaaagaaatgtatggttcagatttcatactttcggaatcagtgagtcgatgcgcaaaagcgatcttcagaggtattggtagaggagaggcatacgtcatagagccatcttggataaaatggatctttttgataaaaaatgtttgtcctgagatcgttgactacttactcgactatatatttgttagttatctaaaaccttatttcaagcgtgat

SEQ ID NO: 5 - protein, 10 aa, Oryza sativa
MEQVVNAVLD

SEQ ID NO: 6 - DNA, 30 bp, Oryza sativa
atggagcaggtggtgaacgcggtgctggac

SEQ ID NO: 7 - protein, 312 aa, Oryza sativa
ENMHNKVVLITGASSAIGEQIAYEYARRNANLVLVARREHRLFAVRENARALGAGQVLVIAADVVKEDDCRRLVGDTISFFGQLNHLVNTVSLGHDFCFEEAGDTVAFPHLMDVNFWGNVYPTYAALPYLRRSHGRVVVNAAVEIWLPMPRMTLYSAAKAAVIDFYESLRYEVGDEVGISVATHGWIGGEASGGKFMLEEGAEMQWKGEEREVPLAGGQVEAYARMVVAGACRGDAHVKHPNWYDVFLVFRAFAPDVLAWTFRLLLSTPSPSPPASARRHQLAALPAPPLHPLLEYPSARSPGRAAQQHKLE

SEQ ID NO: 8 - DNA, 936 bp, Oryza sativa
gagaacatgcacaacaaggtcgtcctcatcaccggcgcctcctccgccatcggagagcaaatcgcgtacgagtacgcgcggaggaacgcgaacctggtgctggtggcgaggcgggagcaccggctgttcgcggtgcgggagaacgcgcgggccctcggcgccggccaggtcctcgtcatcgccgccgacgtcgtcaaggaggacgactgccggaggctcgtcggcgacaccatcagcttcttcggccagttaaaccatctggtgaacacggtgagcttgggccacgatttctgcttcgaggaggccggcgacacggtcgcgttccctcacctcatggacgtcaacttctgggggaacgtgtacccgacgtacgccgcgctcccctacctgcggcggagccatggccgtgtcgtcgtcaacgccgccgtcgagatctggctgcccatgcccaggatgaccctctactctgcggccaaggcggcggtgatcgatttctacgagtcgctccggtacgaggtgggcgatgaggtgggcatcagcgtggcgacgcacggctggatcggcggcgaggccagcggcggcaagttcatgctcgaggaaggcgcagagatgcaatggaaaggggaggagcgagaggtgccgctcgccggcgggcaagtggaggcgtacgcgaggatggtggtcgccggcgcgtgccgcggcgacgcccacgtgaagcaccccaactggtacgacgtcttccttgtcttccgcgccttcgcgcccgacgtcctcgcctggacgttccgcctcctgctgtccacgccatcgccgtcgccgccggcgagcgcccgtcgccaccagctcgccgcgctgccggctccgccgctccacccgctgctcgagtacccgtcggctcggagccccgggcgcgccgcccagcagcacaagctggag

SEQ ID NO: 9 - protein, 9 aa, Populus trichocarpa
MSFINSVLD

SEQ ID NO: 10 - DNA, 27 bp, Populus trichocarpa
atgagttttataaactctgtgttggat

SEQ ID NO: 11 - protein, 284 aa, Populus trichocarpa
EDMEDKVVIITGASSGIGEQIAYEYAKRKAILVLIARREHRLRGVSEKARYIGAKRVLIMAADVVKEDDCRRFVNETINYFGRVDHLVNTASLGHTFYFEEVGDTSVFPHFLDINFWGNVYPTYVALPYLRQSNGRVVVNAAVESWLPLPRMSLYAAAKAALVSFYESLRFEVNGEVGITIASHGWIGSEMSRGKFMLEDGAEMQWKEEREVNGTGGPVEDYAKMIVSGACRGHQYVKYPSWYDIFLLYRMFAPGILNWALRMLLAPNGSRRTSMIGTGRPALI

SEQ ID NO: 12 - DNA, 852 bp, Populus trichocarpa
gaggatatggaggataaagttgttatcatcactggagcttcttctggcataggagaacaaattgcatatgaatatgcaaagaggaaagcaattcttgttctgattgcacgtagagagcaccggcttagaggggtcagtgagaaagctaggtatattggtgcaaagcgtgtcctgattatggctgcagatgttgtcaaggaggatgattgtaggagatttgtcaatgagaccataaattactttggtcgggtggatcatcttgtcaatacagcaagtttggggcatacattttactttgaagaagtaggagacacctctgtgtttccccatttcttggacataaacttttggggaaatgtctatccaacttatgtggctcttccatacctacgtcagagcaatggacgagttgttgttaatgcagcagttgagagctggttacctctgccgagaatgagcttatatgctgctgcaaaggctgccctggtgagcttctacgagtcactgagatttgaagtgaatggtgaagttggaataacaattgcatctcatggttggattgggagcgaaatgagtagaggcaagttcatgctagaggatggagcagagatgcaatggaaagaagagagagaagtaaacgggactggtggtccagtagaggactatgcaaagatgattgtgtcgggagcttgccgaggacatcaatatgtcaagtacccaagctggtatgacatattcctcctttacaggatgtttgcacctggaattctcaactgggctcttcgaatgttgcttgcaccgaatggttcaagaagaacgtctatgataggcaccgggagacctgcattaatt

SEQ ID NO: 13 - protein, 9 aa, Selaginella moellendorffii
MDLIQMLLN

SEQ ID NO: 14 - DNA, 27 bp, Selaginella moellendorffii
atggatttgatacaaatgctgcttaat

SEQ ID NO: 15 - protein, 304 aa, Selaginella moellendorffii
ENVRGKVAVITGASSGIGEYMAYEYGKRGAKVVLCGRRENQLKNVQERVGSEGATDTLVVVADVSREEECKKVVDETINTLGKIDHLVCNHGIANSFFVEEAKGLEIFRKIMDVNFMGCVYTTYFALPHLRKSRGKIVVTASTASWLPIPRMSIYNASKAAVVNFFDTLRTELRSDIGGMTIAMPGYIHSEMTMGKFMSAEGKHDMNVDIRDTLVGPSPVASTQYCAKQIVSAVTRGERYVVVPTWYKVSLLFRVFVPQLLETFISLLFVKELQPGKPVTKVIMDSFPSAEKVLYPPGMQQKTD

SEQ ID NO: 16 - DNA, 912 bp, Selaginella moellendorffii
gaaaatgtccgcggcaaggtcgccgtcatcactggggcttcttccgggattggagagtacatggcttatgagtatggcaagcgtggagccaaggtggtgctttgtgggaggcgagagaatcaactcaagaacgtccaggagcgcgttggatccgagggtgctactgatactctggtggttgtcgccgacgtttctagggaggaagaatgtaagaaggttgtggatgagacgatcaacaccttgggaaaaattgatcaccttgtctgcaatcatggtattgcgaacagcttcttcgtcgaggaagcgaaaggtttggagatcttccgcaagatcatggatgtgaatttcatgggttgcgtctatactacatacttcgctttgcctcatctccgcaagagccgaggaaagattgtcgtcacggcctcgactgcgtcctggcttcccatcccgaggatgtcaatctacaacgcctccaaagcagccgtggtgaacttcttcgataccctgaggactgagcttcgatcagatataggaggaatgacgattgccatgcctggctacatccacagcgagatgacaatgggcaagttcatgtcagccgaggggaagcacgacatgaacgttgacatacgcgacactctcgtcggcccttctccagtagcttcaacgcagtactgcgcgaagcagatcgtgtccgcggtcaccaggggcgagcgctacgtggtggtgccgacctggtacaaggtctcactcctgtttcgcgtcttcgttccccagctcctggagactttcataagcctcttgttcgtcaaggagctacagccagggaagccagtcaccaaagtcatcatggacagcttcccgagcgccgagaaagtcttgtatccccctggaatgcagcagaagacagac

SEQ ID NO: 17 - protein, 9 aa, Physcomitrella patens
MGFLGSFLN

SEQ ID NO: 18 - DNA, 27 bp, Physcomitrella patens
atgggattccttggaagcttcctcaac

SEQ ID NO: 19 - protein, 299 aa, Physcomitrella patens
EDMRGKVVLITGASSGIGEHIAYEYAKKGARLALVGRRENLLMEVADRAITRGASDVKVLVGDVTKEADCKRFLEETIQKYGRLDHLVNNAGVAHSFFFSETKDLKALTSTLDTTFWGQVYMTYFAIPHLRRTHGKVLVMASTASWLPYPRQTLYNAGKAGVLAFFDTLRVEVGDVIGITIVMPGWIESEITKGKFIHEDGDIWTDQMERDMHIGPVPVTSVTECANAAVKGVIRGSHYVTVPFYYSAFLLYRMFAPEVLDWIFRLIFVKHPQKPLSKQVLKASEVHRVLYPTSIQKAD

SEQ ID NO: 20 - DNA, 897 bp, Physcomitrella patens
gaggacatgcgaggaaaagtcgtactaatcacaggagcatcatctggaattggagagcacatagcgtatgagtacgcgaagaagggggctcgcctcgcgctcgtgggacggcgggagaatttgctcatggaggttgcagacagggcgataacgagaggggcatcagatgtgaaggtcttagtcggagatgtcacgaaagaggcagactgcaaacgttttctagaagaaacgattcaaaaatatggccggctggaccatctggtgaacaacgctggagttgctcacagcttcttctttagcgagacaaaggatttgaaggcgctgacatcaactttggacacaacattctggggtcaagtgtacatgacgtactttgcaattccgcatttgcggcgcacacatggcaaggttctcgttatggcgtctactgcgagctggctgccatatccacggcaaacactatacaatgctggaaaggcaggtgtgttagctttctttgacacattacgagtagaggttggtgacgtaattggtatcaccatagtcatgccgggatggattgaaagcgagatcactaaaggcaaatttatacatgaagatggcgacatatggacagaccagatggaacgagacatgcatatcggtccagtgcccgtgacatcggtaactgaatgtgccaacgctgcagtgaagggcgttattagaggctcccattatgtgacggttccattttactactctgcgtttctcttataccgaatgtttgctcctgaagtcttggattggatattccgacttatttttgttaaacatccccagaagcctctgagcaaacaggtactgaaagcctcagaggttcacagggttttgtacccaacaagtattcagaaggcagat

SEQ ID NO: 21 - protein, 40 aa, Arabidopsis thaliana
MELINDFLNLTAPFFTFFGLCFFLPPFYFFKFLQSIFSTI

SEQ ID NO: 22 - DNA, 120 bp, Arabidopsis thaliana
atggagttgataaacgactttctcaacctaactgctcctttcttcaccttctttggtctctgcttcttcttgcctcctttttacttcttcaagttcttgcagtctattttctcgacaatt

SEQ ID NO: 23 - protein, 40 aa, Sesamum indicum
MDLIHTFLNLIAPPFTFFFLLFFLPPFQIFKFFLSILGTL

SEQ ID NO: 24 - DNA, 120 bp, Sesamum indicum
atggatctaatccacactttcctcaacttaatagctccccctttcaccttcttcttccttctctttttcttgccacccttccagattttcaagttcttcctttcaatcttgggcaccctt

SEQ ID NO: 25 - protein, 346 aa, Arabidopsis thaliana
MELINDFLNLTAPFFTFFGLCFFLPPFYFFKFLQSIFSTIFSENLYGKVVLITGASSGIGEQLAYEYACRGACLALTARRKNRLEEVAEIARELGSPNVVTVHADVSKPDDCRRIVDDTITHFGRLDHLVNNAGMTQISMFENIEDITRTKAVLDTNFWGSVYTTRAALPYLRQSNGKIVAMSSSAAWLTAPRMSFYNASKAALLSFFETMRIELGGDVHITIVTPGYIESELTQGKYFSGEGELIVNQDMRDVQVGPFPVASASGCAKSIVNGVCRKQRYVTEPSWFKVTYLWKVLCPELIEWGCRLLYMTGTGMSEDTALNKRIMDIPGVRDYKDDDDKENLYF

SEQ ID NO: 26 - protein, 7 aa, Artificial Sequence
  Synthetic linker
ENLYFQS

SEQ ID NO: 27 - DNA, 21 bp, Artificial Sequence
  Synthetic linker
gagaacctctacttccaatcg

SEQ ID NO: 28 - protein, 198 aa, Artificial Sequence
  Synthetic linker
CITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHA

SEQ ID NO: 29 - protein, 594 aa, Artificial Sequence
  Synthetic linker
TGCATCACGGGAGATGCACTAGTTGCCCTACCCGAGGGCGAGTCGGTACGCATCGCCGACATCGTGCCGGGTGCGCGGCCCAACAGTGACAACGCCATCGACCTGAAAGTCCTTGACCGGCATGGCAATCCCGTGCTCGCCGACCGGCTGTTCCACTCCGGCGAGCATCCGGTGTACACGGTGCGTACGGTCGAAGGTCTGCGTGTGACGGGCACCGCGAACCACCCGTTGTTGTGTTTGGTCGACGTCGCCGGGGTGCCGACCCTGCTGTGGAAGCTGATCGACGAAATCAAGCCGGGCGATTACGCGGTGATTCAACGCAGCGCATTCAGCGTCGACTGTGCAGGTTTTGCCCGCGGGAAACCCGAATTTGCGCCCACAACCTACACAGTCGGCGTCCCTGGACTGGTGCGTTTCTTGGAAGCACACCACCGAGACCCGGACGCCCAAGCTATCGCCGACGAGCTGACCGACGGGCGGTTCTACTACGCGAAAGTCGCCAGTGTCACCGACGCCGGCGTGCAGCCGGTGTATAGCCTTCGTGTCGACACGGCAGACCACGCGTTTATCACGAACGGGTTCGTCAGCCACGCT

SEQ ID NO: 30 - DNA, 496 bp, Artificial Sequence
  Synthetic - promoter Hsp70A-Rbc52
tcgctgaggcttgacatgattggtgcgtatgtttgtatgaagctacaggactgatttggcgggctatgagggcgggggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaa